SEQUENCE LISTING (13 sequences)

SEQ ID NO: 1 - DNA, 12 bp, synthetic construct
  source 1..12: mol_type = other DNA
  misc_feature 1: note = The thymine residue of SEQ ID NO: 1 located at position 1 is attached to thymine residue of SEQ ID NO: 11 located at position 12 via iSpC12 spacer.
tacatcagagat

SEQ ID NO: 2 - DNA, 23 bp, synthetic construct
  source 1..23: mol_type = other DNA
tcctcgggcctactgcctcaccg

SEQ ID NO: 3 - DNA, 35 bp, synthetic construct
  source 1..35: mol_type = other DNA
aggcccgaggattaacccacgccgaatcctagact

SEQ ID NO: 4 - DNA, 23 bp, synthetic construct
  source 1..23: mol_type = other DNA
ctgggctgcacgctactcaccag

SEQ ID NO: 5 - DNA, 35 bp, synthetic construct
  source 1..35: mol_type = other DNA
gtgcagcccagttaacccacgccgaatcctagact

SEQ ID NO: 6 - DNA, 23 bp, synthetic construct
  source 1..23: mol_type = other DNA
gttctcaaagtatctctgatgta

SEQ ID NO: 7 - DNA, 35 bp, synthetic construct
  source 1..35: mol_type = other DNA
actttgagaacttaacccacgccgaatcctagact

SEQ ID NO: 8 - DNA, 24 bp, synthetic construct
  source 1..24: mol_type = other DNA
  misc_feature 1: note = The thymine residue of SEQ ID NO: 8 located at position 1 is attached to guanine residue of SEQ ID NO: 12 located at position 24 via iC9Sp spacer.
ttaacccacgccgaatcctagact

SEQ ID NO: 9 - DNA, 24 bp, synthetic construct
  source 1..24: mol_type = other DNA
  misc_feature 1: note = The adenine residue of SEQ ID NO: 9 located at position 1 is attached to guanine residue of SEQ ID NO: 13 located at position 24 via iSpC12 spacer.
agtctaggattcggcgtgggttaa

SEQ ID NO: 10 - DNA, 12 bp, synthetic construct
  source 1..12: mol_type = other DNA
  misc_feature 12: note = The cytosine residue of SEQ ID NO: 10 located at position 12 is attached to cytosine residue of SEQ ID NO: 11 located at position 1 via iSpC12 spacer.
ctggtgagtagc

SEQ ID NO: 11 - DNA, 12 bp, synthetic construct
  source 1..12: mol_type = other DNA
  misc_feature 1: note = The cytosine residue of SEQ ID NO: 11 located at position 1 is attached to cytosine residue of SEQ ID NO: 10 located at position 12 via iSpC12 spacer.
  misc_feature 12: note = The thymine residue of SEQ ID NO: 11 located at position 12 is attached to thymine residue of SEQ ID NO: 1 located at position 1 via iSpC12 spacer.
cggtgaggcagt

SEQ ID NO: 12 - DNA, 24 bp, synthetic construct
  source 1..24: mol_type = other DNA
  misc_feature 24: note = The guanine residue of SEQ ID NO: 12 located at position 24 is attached to thymine residue of SEQ ID NO: 8 located at position 1 via iC9Sp spacer.
caaagtagtctaggattcggcgtg

SEQ ID NO: 13 - DNA, 24 bp, synthetic construct
  source 1..24: mol_type = other DNA
  misc_feature 24: note = The guanine residue of SEQ ID NO: 13 located at position 24 is attached to adenine residue of SEQ ID NO: 9 located at position 1 via iSpC12 spacer.
cacgccgaatcctagactactttg

(19) United States
(12) Patent Application Publication
(10) Pub. No.: US 2026/0103745 A1
(43) Pub. Date: Apr. 16, 2026

(54) METHOD AND SYSTEM FOR MULTIPLEXED DETECTION OF ONE OR MORE ALLELES IN A SAMPLE

(71) Applicant: Giner, Inc., Newton, MA (US)

(72) Inventors: Muhit Rana, Southborough, MA (US); Avni A. Argun, Newton, MA (US)

(21) Appl. No.: 19/309,032

(22) Filed: Aug. 25, 2025

Related U.S. Application Data
(60) Provisional application No. 63/687,437, filed on Aug. 27, 2024.

(51) Int. Cl. C12Q 1/6827; B01L 3/00

(52) U.S. Cl. CPC C12Q 1/6827; B01L 3/50273; B01L 2200/026; B01L 2300/041; B01L 2300/0645; B01L 2400/0487

(57) ABSTRACT

Method and system for detecting one or more alleles of interest in a sample. In one embodiment, the method includes providing a multi-welled plate, each well having an electrochemical sensing element and a capture probe. The capture probe includes two or more nucleotide capture sequences, each designed to hybridize specifically with an allele of interest. The capture probe is exposed to a biofluid sample so that any alleles of interest present bind to the capture probe. Next, an initiator that is specific to only one of the alleles of interest is added to the well. If the allele of interest specific to the initiator is bound to the capture probe, the initiator binds to the allele of interest. Next, hairpin molecules that undergo hybridization chain reaction when the initiator is present are added. The hairpin molecules are coupled to a tag. The presence of the tag is then detected.

### CROSS-REFERENCE TO RELATED APPLICATIONS

The present application claims the benefit under 35 U.S.C. 119 (e) of U.S. Provisional Patent Application No. 63/687,437, inventors Muhit Rana et al., filed Aug. 27, 2024, the disclosure of which is incorporated herein by reference.

### FEDERALLY SPONSORED RESEARCH OR DEVELOPMENT

This invention was made with government support under W81XWH21P0007 awarded by the Department of Defense (DoD). The government has certain rights in the invention.

### SEQUENCE LISTING

The instant application contains a Sequence Listing XML which has been submitted electronically in XML file format and is hereby incorporated by reference in its entirety. Said XML copy, created on Aug. 22, 2025, is named 84750A and is 16,655 bytes in size.

### BACKGROUND OF THE INVENTION

The present invention relates generally to the detection of alleles (i.e., different versions of a gene) in a sample and relates more particularly to a novel method and system for detecting alleles in a sample.

Cytochrome P450 enzymes (CYPs) are a family of naturally-occurring enzymes that not only are involved in the production of compounds like cholesterol, steroids, prostacyclin and thromboxane A2 but also are essential in the detoxification and metabolism of prescription drugs and other foreign chemicals. (See Lynch et al., “The effect of cytochrome P450 metabolism on drug response, interactions, and adverse effects,” Am Fam Physician, 76 (3): 391-6 (2007), which is incorporated herein by reference.) The expression of genes for multiple CYPs, including the genes for cytochrome P450 enzymes CYP2D6 and CYP2C19, in the liver and the small intestine, is responsible for 90 percent of the metabolism of medications currently in use. (See Slaughter et al., “Recent advances: the cytochrome P450 enzymes,” Ann Pharmacother, 29 (6): 619-24 (1995); and Wilkinson, “Drug metabolism and variability among patients in drug response,” N Engl J Med, 352 (21): 2211-21 (2005), both of which are incorporated herein by reference.)

Due to the role of CYPs in drug metabolism, the genetic variants of CYPs, particularly single nucleotide polymorphisms of CYPs, have been estimated to account for 20 to 30 percent of drug response variability in patients that receive common prescription drugs ranging from beta blockers and antidepressants to opioids and proton pump inhibitors (PPI). (See Sim et al., “Pharmacogenomics of drug-metabolizing enzymes: a recent update on clinical implications and endogenous effects,” Pharmacogenomics J, 13 (1): 1-11 (2013); and Abubakar et al., “Precision medicine and pharmacogenomics in community pharmacy and primary care settings,” Pharmacy Today, 24:55-68 (2018), both of which are incorporated herein by reference.) The dominant single nucleotide polymorphisms of some CYPs, such as the CYP2D6*1 and CYP2D6*10 variants of CYP2D6 and the CYP2C19*3 variant of CYP2C19, have been frequently reported in clinical reports as having an altered enzymatic activity as compared to the corresponding wild-type enzyme. (See Hicks et al., “Challenges in CYP2D6 phenotype assignment from genotype data: a critical assessment and call for standardization,” Curr Drug Metab, 15 (2): 218-32 (2014), which is incorporated herein by reference.)

Consequently, identifying the presence of a particular allele of one or more CYPs in a patient can provide valuable guidance for adjusting medication prescriptions and therapy plans from a pharmacogenetic aspect. In view of the fact that 18% of prescribed drugs in the United States carry actionable pharmacogenetic (PGx) labels (see Relling et al., “Pharmacogenomics in the clinic,” Nature, 526 (7573): 343-50 (2015), which is incorporated herein by reference), there is undoubtedly an unmet need for a rapid, economic, and easy-to-use diagnostic capable of detecting a CYP single nucleotide polymorphism in a sample.

Hybridization chain reaction (HCR) is an enzyme-free, nucleic acid reaction, in which two different types of DNA hairpin molecules hybridize to one another to form a long, double-stranded DNA extension but only when triggered by the presence of a complementary single-stranded DNA molecule that is termed “the initiator.” (See Dirks et al., “Triggered amplification by hybridization chain reaction,” Proc Natl Acad Sci USA, 101:15275-8 (2004), which is incorporated herein by reference.) More specifically, when the initiator is added to a mixture of two different types of DNA hairpin molecules, the initiator opens the first type of DNA hairpin molecule (sometimes referred to herein as “the first hairpin molecule”) and hybridizes to a complementary sequence of the first hairpin molecule. The now linear first hairpin molecule then immediately opens the second type of DNA hairpin molecule (sometimes referred to herein as “the second hairpin molecule”) and hybridizes to a complementary sequence of the second hairpin molecule. The now linear second hairpin molecule then opens and hybridizes to another copy of the first hairpin molecule, which, in turn, then opens and hybridizes to another copy of the second hairpin molecule, and so on. The repeated opening and hybridization of the first and second hairpin molecules eventually results in a long double-stranded extension that amplifies the presence of a detection target and that is detectable using, for example, an enzyme-based colorimetric assay.

Considerable advancements in highly-sensitive detection methods, such as microarray analysis and high-throughput sequencing, have enabled the detection and monitoring of biomarkers that were previously too low in concentration to be detected. These technological breakthroughs have not only facilitated the tracking of disease progression and categorization (Michaels et al., “Precise tuning of gene expression levels in mammalian cells,” Nat Commun, 10(1):818 (2019); and Schmidt, “Introducing Single-Nucleotide Polymorphism Markers in the Diagnosis of Wilson Disease,” Clin Chem, 53 (9): 1568-9 (2007), both of which are incorporated herein by reference), but also have illuminated a path to developing more precise and personalized treatment plans for a variety of medical conditions, including inflammatory responses (Holcomb et al., “Host-Based Peripheral Blood Gene Expression Analysis for Diagnosis of Infectious Diseases,” J Clin Microbiol, 55 (2): 360-8 (2017), which is incorporated herein by reference), malignant tumors, and drug reactions (Abubakar et al., “Precision medicine and pharmacogenomics in community pharmacy and primary care settings,” Pharmacy Today, 24:55-68 (2018)). However, because many of these types of detection methods, such as microarray analysis, require intensive professional labor and expensive equipment, such methods are still a long way from being utilized in clinical practice.

### SUMMARY OF THE INVENTION

It is an object of the present invention to provide a novel method and system for detecting one or more alleles of interest in a sample.

It is another object of the present invention to provide a method and system as described above that address at least some of the shortcomings associated with existing methods.

Therefore, according to one aspect of the invention, there is provided a method for detecting the presence of one or more alleles of interest in a sample, the method comprising the steps of (a) providing an immobilized capture probe on a support, the immobilized capture probe comprising a first nucleotide capture sequence and a second nucleotide capture sequence, the first nucleotide capture sequence designed to hybridize specifically with a first portion of a first allele of interest, the second nucleotide capture sequence designed to hybridize specifically with a first portion of a second allele of interest, the second allele of interest being different from the first allele of interest; (b) exposing the immobilized capture probe to a sample and removing any non-specific binding, whereby, if the sample contains the first allele of interest, the first portion of the first allele of interest specifically hybridizes to the first nucleotide sequence, and/or if the sample contains the second allele of interest, the first portion of the second allele of interest specifically hybridizes to the second nucleotide sequence, whereas, if the sample contains neither the first allele of interest nor the second allele of interest, nothing specifically hybridizes to the first nucleotide sequence or the second nucleotide sequence; (c) exposing the immobilized capture probe, and any first and second alleles of interest bound thereto, to a first initiator and removing any non-specific binding, wherein a first portion of the first initiator is designed to hybridize specifically to one of a second portion of the first allele of interest and a second portion of the second allele of interest; (d) exposing the immobilized capture probe, and any initiator bound thereto, to first and second hairpin molecules and removing any non-specific binding, wherein the first and second hairpin molecules are designed to undergo a hybridization chain reaction when actuated by the first initiator, and wherein at least one of the first and second hairpin molecules is coupled to a tag; and (e) determining the presence of the tag.

In a more detailed feature of the invention, the one or more alleles of interest may include one or more alleles of cytochrome P450 genes.

In a more detailed feature of the invention, the cytochrome P450 genes may include one or more of CYP2D6 and CYP2C9.

In a more detailed feature of the invention, the one or more alleles of interest may be selected from the group consisting of an allele containing a CYP2D6*1 single nucleotide polymorphism, an allele containing a CYP2D6*10 single nucleotide polymorphism, and an allele containing a CYP2C19*17 single nucleotide polymorphism.

In a more detailed feature of the invention, the immobilized capture probe may further comprise a third nucleotide capture sequence, the third nucleotide capture sequence may be designed to hybridize specifically with a first portion of a third allele of interest, the third allele of interest may be different from the first and second alleles of interest, and the initiator may be designed to hybridize specifically to one of a second portion of the first allele of interest, a second portion of the second allele of interest, and a second portion of the third allele of interest.

In a more detailed feature of the invention, the support may comprise a first well, and at least one copy of the immobilized capture probe may be disposed in the first well.

In a more detailed feature of the invention, the support may further comprise a second well, and the method may further comprise repeating steps (a) through (e) for the second well, except that a second initiator may be used instead of the first initiator in the second well, and the second initiator may be designed to hybridize specifically to the other of the second portion of the first allele of interest and the second portion of the second allele of interest.

In a more detailed feature of the invention, the support may comprise an electrochemical sensing element.

In a more detailed feature of the invention, the support may further comprise a well, and the electrochemical sensing element may be associated with the well.

In a more detailed feature of the invention, the step of determining the presence of the tag may comprise exposing the tag to an enzyme-linked antibody that may be designed to specifically bind to the tag and removing any non-specific binding, exposing the enzyme-linked antibody to a substrate that undergoes a detectable change when exposed to the enzyme-linked antibody, and detecting the detectable change in the substrate.

In a more detailed feature of the invention, the detectable change in the substrate may be determined by optical analysis.

In a more detailed feature of the invention, the detectable change in the substrate may be determined by electrochemical analysis.

In a more detailed feature of the invention, the first hairpin molecule may include a first hairpin nucleotide sequence, the second hairpin molecule may include a second hairpin nucleotide sequence, and the first hairpin molecule may further include a first spacer at or near a midpoint of the first hairpin nucleotide sequence and/or the second hairpin molecule may further include a second spacer at or near a midpoint of the second hairpin nucleotide sequence.

According to another aspect of the invention, there is provided a method for detecting the presence of a cytochrome P450 allele of interest in a sample, the method comprising the steps of (a) providing an immobilized capture probe on a support, the immobilized capture probe comprising a first nucleotide capture sequence, the first nucleotide capture sequence designed to hybridize specifically with a first portion of a cytochrome P450 allele of interest; (b) exposing the immobilized capture probe to a sample and removing any non-specific binding, whereby, if the sample contains the cytochrome P450 allele of interest, the first portion of the cytochrome P450 allele of interest specifically hybridizes to the first nucleotide sequence whereas, if the sample does not contain the cytochrome P450 allele of interest, nothing specifically hybridizes to the first nucleotide sequence; (c) exposing the immobilized capture probe, and any cytochrome P450 allele of interest bound thereto, to a first initiator and removing any non-specific binding, wherein a first portion of the first initiator is designed to hybridize specifically to the cytochrome P450 allele of interest; (d) exposing the immobilized capture probe, and any first initiator bound thereto, to first and second hairpin molecules and removing any non-specific binding, wherein the first and second hairpin molecules are designed to undergo a hybridization chain reaction when actuated by the first initiator, and wherein at least one of the first and second hairpin molecules is coupled to a tag; and (e) determining the presence of the tag.

In a more detailed feature of the invention, the cytochrome P450 allele of interest may be an allele of one of CYP2D6 and CYP2C9.

In a more detailed feature of the invention, the cytochrome P450 allele of interest may be selected from the group consisting of an allele containing a CYP2D6*1 single nucleotide polymorphism, an allele containing a CYP2D6*10 single nucleotide polymorphism, and an allele containing a CYP2C19*17 single nucleotide polymorphism.

In a more detailed feature of the invention, the support may comprise a first well and an electrochemical sensing element associated with the well, and the immobilized capture probe may be disposed in the first well.

In a more detailed feature of the invention, the step of determining the presence of the tag may comprise exposing the tag to an enzyme-linked antibody that may be designed to specifically bind to the tag and removing any non-specific binding, exposing the enzyme-linked antibody to a substrate that undergoes a detectable change when exposed to the enzyme-linked antibody, and detecting the detectable change in the substrate.

In a more detailed feature of the invention, the detectable change in the substrate may be determined by optical analysis.

In a more detailed feature of the invention, the detectable change in the substrate may be determined by electrochemical analysis.

According to still another aspect of the invention, there is provided a system for use in electrochemically detecting an allele of interest in a sample, the system comprising (a) a cartridge assembly, the cartridge assembly comprising (i) a cartridge, the cartridge comprising one or more wells and further comprising a corresponding electrochemical sensing element associated with each of the one or more wells, and (ii) a cover, the cover being removably mounted on the cartridge, the cover being shaped to include one or more fluid conduits in fluid communication with the one or more wells; and (b) a reader for electrochemically detecting the presence of an analyte of interest in each of the one or more wells, the analyte of interest being indicative of the presence of the allele of interest, the reader comprising (i) a potentiostat; (ii) one or more electrical connectors for electrically connecting the one or more electrochemical sensing elements to the potentiostat; (iii) a control unit operably coupled to the potentiostat for operating the potentiostat and for analyzing signals generated by the potentiostat; (iv) an output device operably coupled to the control unit for outputting results of analysis performed by the control unit; (v) a pump, the pump being operably coupled to the control unit; (vi) one or more fluid connectors operably coupled to the one or more fluid conduits in the cover and to the pump; (vii) a fluid waste receptacle operably coupled to the pump for receiving fluid removed from the one or more wells by the pump; and (viii) a power supply.

In a more detailed feature of the invention, the potentiostat may have one or more modules, and each module may correspond to a different one of the one or more electrochemical sensing elements.

In a more detailed feature of the invention, the system may further comprise a foot pedal, and the foot pedal may be operably coupled to the control unit to actuate operation of the pump.

In a more detailed feature of the invention, each electrochemical sensing element may comprise a working electrode, a counter electrode and a reference electrode.

In a more detailed feature of the invention, one or more of the working electrode, the counter electrode and the reference electrode may be screen-printed using a carbon ink.

In a more detailed feature of the invention, the output device may be a monitor.

In a more detailed feature of the invention, the system may further comprise a housing, the housing may be shaped to include a shelf, and the shelf may be configured to matingly receive the cartridge assembly in such a way as to connect the one or more fluid conduits of the cover to the one or more fluid connectors of the reader and to connect the one or more electrochemical sensing elements of the cartridge to the one or more electrical connectors of the reader.

Additional objects, as well as aspects, features, and advantages, of the present invention will be set forth in part in the description which follows, and in part will be obvious from the description or may be learned by practice of the invention. In the description, reference is made to the accompanying drawings which form a part thereof and in which is shown by way of illustration various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. The drawings are not necessarily drawing to scale, and certain components may have undersized and/or oversized dimensions for purposes of explication. In the drawings wherein like reference numeral represents like parts:

FIG. 1 is a flowchart illustrating one embodiment of a method for detecting one or more alleles of interest in a sample according to the present invention;

FIG. 2 is a schematic representation of the method shown in FIG. 1;

FIG. 3 is a schematic diagram of one embodiment of a system for use in detecting one or more alleles of interest in a sample according to the present invention;

FIG. 4 is a perspective view of one embodiment of a physical manifestation of the reader shown in FIG. 3;

FIG. 5 is a perspective view of the reader shown in FIG. 4, with certain components not being shown for simplicity and/or clarity;

FIG. 6 is a partly exploded fragmentary perspective view of the reader shown in FIG. 4, together with a cartridge assembly;

FIG. 7 is an illustration of one embodiment of a graphical user interface suitable for use in the reader shown in FIG. 4;

FIGS. 8A and 8B are images of the electrophoresis gel results obtained in Example 3;

FIG. 9 is a graphic representation of the results of the presence test discussed in Example 4;

FIGS. 10A and 10B are graphic representations of the results of the buffer optimization tests discussed in Example 5;

FIGS. 11A and 11B are graphic representations of the results of the sensitivity tests discussed in Example 6 for the alleles CYP2D6*10 and CYP2D6*1, respectively;

FIG. 11C is a graphic representation of electrochemical readings discussed in Example 6 for the detection of the CYP2D6*1 allele;

FIG. 12 is a graphic representation of the results of the sensitivity test discussed in Example 6 for the detection of the double-stranded CYP2D6*10 allele;

FIG. 13 is a graphic representation of the results of the specificity test discussed in Example 7;

FIG. 14 is a graphic representation of the optical density at 450 nm of an Anti-DIG-HRP antibody dilution data set detected using bench top plate reader, as described in Example 8; and

FIG. 15 is a graphic representation of electrochemical readings of an Anti-DIG-HRP antibody dilution data set detected using an EmStat4M-equipped prototype reader and an EmStat3M-equipped PC, as described in Example 8.

### DETAILED DESCRIPTION OF THE INVENTION

As noted above, single nucleotide polymorphisms (SNPs) of cytochrome P450 genes, such as CYP2D6 and CYP2C9, are linked to the metabolization rate of many clinically used drugs (˜25%). Detection of these mutant alleles is essential to assess drug metabolism and is instrumental in guiding drug therapy and dosage adjustment. The present inventors believe that the growing importance of pharmacogenetic testing highlights the need for efficient multiplexed testing to detect the SNPs in multiple genes of interest from a single sample.

To this end, the present invention is directed, at least in part, at a novel technique for detecting a desired allele of interest, wherein said technique does not rely on polymerase chain reaction (PCR). Instead, the present technique utilizes hybridization chain reaction (HCR) and provides a compact and cost-effective solution that may be used at the point of care (POC) to detect one or more gene biomarkers in biofluids, such as, but not limited to, blood. Moreover, in at least one embodiment, the present invention may be used for multiplexed detection of SNPs in one or more genes of interest from a single sample or from multiple samples. Such a gene of interest may be a cytochrome P450 gene, such as CYP2D6 and CYP2C9.

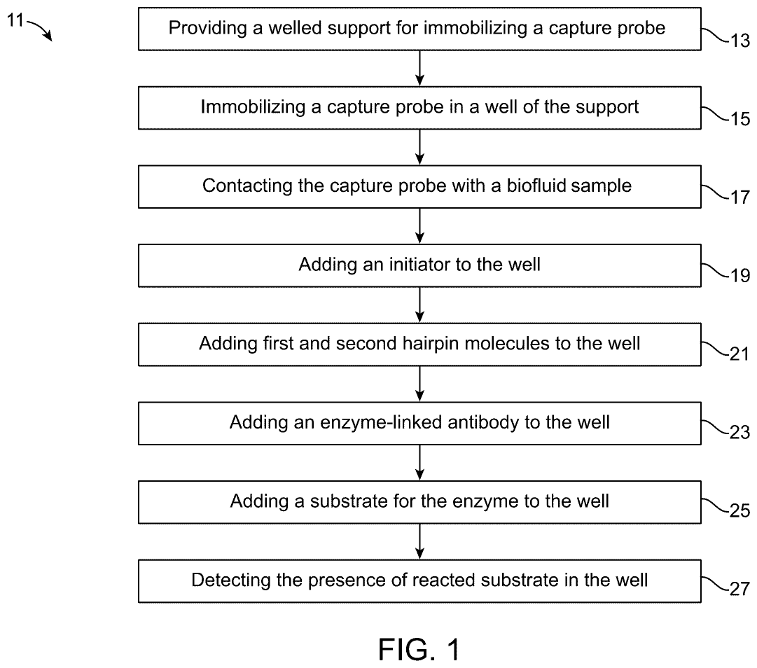

Referring now to FIG. 1, there is shown a flowchart illustrating one embodiment of a method for detecting a desired allele of interest according to the present invention, the method being represented generally by reference numeral 11. Details of method 11 that are discussed elsewhere in this application or that are not critical to an understanding of the invention may be omitted from FIG. 1 and/or from the accompanying description herein or may be shown in FIG. 1 and/or described herein in a simplified manner.

Method 11 may comprise a step 13 of providing a support. The support may comprise a structure that is suitable for immobilizing a capture probe. In at least one embodiment, the support may comprise, for example, a structure comprising one or more wells. For example, according to one embodiment, the support may comprise a 96-well microwell plate. For reasons to become apparent below, one or more of the wells of such a microwell plate may be partially or completely coated with streptavidin or a similar material. According to another embodiment, the support may comprise one or more electrochemical sensing elements. Each electrochemical sensing element, in turn, may comprise, for example, one or more electrodes on a suitable substrate. In at least one embodiment, the one or more electrodes may be screen-printed electrodes, and the one or more of screen-printed electrodes may comprise a working electrode, a counter electrode, and a reference electrode. Each of the screen-printed electrodes may be formed using a carbon ink. In addition, for reasons to become apparent below, one or more of the screen-printed electrodes may be subsequently coated with streptavidin. Where the support comprises one or more electrochemical sensing elements, the support may further comprise a well or fluid volume bounded at an end by said electrochemical sensing element or may have an electrochemical sensing element at least partially positioned therein.

Method 11 may further comprise a step 15 of immobilizing one or more copies of a capture probe on the support. In at least one embodiment, the capture probe may comprise a single-stranded nucleotide sequence that is biotinylated at its 5′-end so that the capture probe may securely bind to the support via biotin-streptavidin binding. In at least one embodiment, the nucleotide sequence of the capture probe may consist of a single nucleotide capture sequence that specifically hybridizes, in part, to an allele of interest; alternatively, the nucleotide sequence of the capture probe may be a multiplexed capture probe that may comprise a plurality of nucleotide capture sequences, each of which may specifically hybridize, in part, to a different allele of interest. For example, in at least one embodiment, the multiplexed capture probe may comprise a single unbranched nucleotide strand comprising three capture sequences, a first of the three capture sequences being a nucleotide sequence specifically hybridizing, in part, to a first allele of interest, a second of the three capture sequences being a nucleotide sequence specifically hybridizing, in part, to a second allele of interest, and a third of the three capture sequences being a nucleotide sequence specifically hybridizing, in part, to a third allele of interest. The foregoing three alleles of interest are preferably different from one another and may be alleles of a single gene or may be alleles of a plurality of genes. For example, in one embodiment, the first capture sequence may consist of or comprise a nucleotide sequence that may specifically hybridize, in part, to an allele containing a CYP2D6*1 single nucleotide polymorphism, the second capture sequence may consist of or comprise a nucleotide sequence that may specifically hybridize, in part, to an allele containing a CYP2D6*10 single nucleotide polymorphism, and the third capture sequence may consist of or comprise a nucleotide sequence that may specifically hybridize, in part, to an allele containing a CYP2C19*17 single nucleotide polymorphism.

In one embodiment, the support may be constructed to comprise a plurality of wells, and each well of the support may have one or more copies of the same type of capture probe immobilized therein, preferably a multiplexed capture probe; however, this need not be the case. For example, in some cases, some wells of a multi-well support may have one or more copies of a multiplexed or non-multiplexed capture probe immobilized therein whereas other wells of the same support may have no capture probe immobilized therein. Alternatively, in some cases, it may be desirable for one or more copies of one type of capture probe to be immobilized in some, but not all, of the wells of a multi-well support and for one or more copies of a different type of capture probe to be immobilized in some or all of the remaining wells of the same multi-well support. Alternatively, in some cases, it may be desirable for one or more copies of a plurality of different types of capture probes to be immobilized in some of the same wells of the same multi-well support.

Immobilization of a capture probe on the support may be effected, for example, by adding one or more copies of the capture probe to a suitable solvent to form a mixture, dispensing a quantity of the mixture into some or all of the wells of the support, allowing, through an incubation period, the capture probe to bind to the support, for example, through biotin-streptavidin binding, and then preferably removing any unbound capture probe and solvent from the support, for example, by washing.

Method 11 may further comprise a step 17 of contacting the capture probe with a suitable biofluid sample. The biofluid sample, which may be obtained from a single individual or which may be pooled from a plurality of individuals, may consist of or comprise blood, plasma, bronchoalveolar (BAL) fluid, saliva, or a fluid derived from or containing one or more of the foregoing. Alternatively, the biofluid sample may consist of or comprise a solvent or carrier that includes a biological specimen obtained from one or more individuals or that has been spiked with or derived from a biological specimen obtained from one or more individuals.

The contacting of the capture probe with the biofluid sample may be effected, for example, by dispensing a quantity of the biofluid sample into one or more of the wells containing the capture probe and allowing one or more target nucleotide sequences that may be present in the biofluid sample to hybridize, in part, to complementary capture sequences of the capture probe. Because hybridization may not readily occur between the capture probe and double-stranded nucleotide sequences in the biofluid sample, the biofluid sample may be pre-treated, prior to being dispensed into the one or more wells, for example, using heat and/or denaturing agents, to denature the double-stranded nucleotide sequences in the biofluid sample into single-stranded nucleotide sequences. After a suitable incubation period to allow the capture probe to capture, if present, a target nucleotide sequence in the sample (with a first portion of the target nucleotide sequence hybridizing with the capture probe and a second portion of the same target nucleotide sequence not hybridizing with the capture probe), the uncaptured portion of the sample may be removed from the one or more wells, for example, by washing.

Method 11 may further comprise a step 19 of adding an initiator to one or more of the wells to which the biofluid sample was added. The initiator of the present invention may consist of or comprise a single-stranded nucleotide sequence that may include a first portion and a second portion, wherein the first portion of the initiator is designed to hybridize specifically to a portion of a captured target nucleotide sequence that did not hybridize to the capture probe, and wherein the second portion of the initiator is designed to hybridize specifically to a hairpin molecule of the type discussed below.

The addition of the initiator to a well may be effected, for example, by adding one or more copies of the initiator to a suitable solvent to form a mixture, dispensing a quantity of the mixture into the one or more wells, allowing, through an incubation period, the initiator to hybridize specifically, in part, to the captured target nucleotide sequence (assuming that such a captured target nucleotide sequence is present), and then preferably removing any unbound initiator and solvent from the one or more wells, for example, by washing.

As can be appreciated, in those cases in which the biofluid sample contains a target nucleotide sequence, the target nucleotide sequence will hybridize, in part, to the capture probe, and the initiator will subsequently hybridize, in part, to the captured target nucleotide sequence. By contrast, in those cases in which the biofluid sample does not contain a target nucleotide sequence, no target nucleotide sequence will hybridize to the capture probe, and, thus, no initiator will hybridize to a captured target nucleotide sequence. As will become apparent from the discussion below, in the former case, a hybridization chain reaction will subsequently occur whereas, in the latter case, no such hybridization chain reaction will occur thereafter.

In those instances in which the support comprises a plurality of wells, each well having the same multiplexed capture probe, different initiators may be used, preferably with only one type of initiator being present in each well containing the multiplexed capture probe, with each type of initiator being designed to specifically hybridize with a different type of captured target nucleotide sequence. For example, in a support having a plurality of wells, each well containing the same multiplexed capture probe having three capture sequences, a first portion of the wells may receive a first initiator, wherein the first initiator specifically hybridizes, in part, to a first type of target nucleotide sequence that has been captured by the first capture sequence of the multiplexed capture probe, a second portion of the wells (i.e., distinct from the first portion of the wells) may receive a second initiator, wherein the second initiator specifically hybridizes, in part, to a second type of target nucleotide sequence that has been captured by the second capture sequence of the multiplexed capture probe, and a third portion of the wells (i.e., distinct from the first and second portions of the wells) may receive a third initiator, wherein the third initiator specifically hybridizes, in part, to a third type of target nucleotide sequence that has been captured by the third capture sequence of the multiplexed capture probe. Consequently, as will become fully apparent below, by using different initiators in different wells, one may achieve multiplexed detection of different target nucleotide sequences using the same capture probe.

Method 11 may further comprise a step 21 of adding multiple copies of first and second hairpin molecules to one or more of the wells. Each of the first and second hairpin molecules may comprise a nucleotide sequence that partially hybridizes with itself to form a double-stranded stem portion and a single-stranded loop portion, the single-stranded loop portion interconnecting the two strands of the stem portion. The 5′-end and 3′-end of the stem portion may each include a single-stranded portion. The first and second hairpin molecules are preferably designed so that the portion of the initiator that does not hybridize to a captured target nucleotide sequence will hybridize specifically to a portion of a first copy of the first hairpin molecule, causing the first copy of the first hairpin molecule to open, which, in turn, will cause the first copy of the first hairpin molecule to hybridize specifically to a portion of a first copy of the second hairpin molecule, causing the first copy of the second hairpin molecule to open, which, in turn, will cause the first copy of the second hairpin molecule to hybridize specifically to a portion of a second copy of the first hairpin molecule, and so on such that multiple copies of the first and second hairpin molecules hybridize to one another in a hybridization chain reaction.

Preferably, one or both of the first hairpin molecule and the second hairpin molecule further comprise a tag or label coupled to the nucleotide sequence. For example, in one embodiment, each of 5′-end and 3′-end of the nucleotide sequence of each of the first hairpin molecule and the second hairpin molecule may be conjugated to a digoxigenin (DIG) molecule. Notwithstanding the above, it is to be understood that tags or labels other than digoxigenin may be used.

The addition of the first and second hairpin molecules to one or more of the wells may be effected, for example, by adding one or more copies of the first and second hairpin molecules to a suitable solvent to form a mixture, dispensing a quantity of the mixture into the one or more wells, allowing, through an incubation period, the first hairpin molecule to hybridize to the initiator, and the first and second hairpin molecules to hybridize to one another in a hybridization chain reaction, and then preferably removing any unbound hairpin molecules and solvent from the one or more wells, for example, by washing.

Method 11 may further comprise a step 23 of adding an enzyme-linked antibody to one or more of the wells in order to bind to the tag or label on one or both of the hairpin molecules. In one embodiment, the enzyme-linked antibody may comprise an anti-DIG antibody that is coupled or conjugated to a horseradish peroxidase (HRP) molecule or complex. As will be discussed further below, horseradish peroxidase may be used to catalyze the oxidation of a substrate, and the occurrence of such oxidation may then be detected. Notwithstanding the above, it is to be understood that enzymes other than horseradish peroxidase may be used.

The addition of the enzyme-linked antibody to one or more of the wells may be effected, for example, by adding one or more copies of the enzyme-linked antibody to a suitable solvent to form a mixture, dispensing a quantity of the mixture into one or more of the wells, allowing, through an incubation period, the enzyme-linked antibody to bind to the tag that is coupled to the first and/or second hairpin molecules, and then preferably removing any unbound enzyme-linked antibody and solvent from the one or more wells, for example, by washing.

Method 11 may further comprise a step 25 of adding to one or more of the wells a substrate for the enzyme of the aforementioned enzyme-linked antibody. In one embodiment, the substrate may comprise 3,3′,5,5′-tetramethylbenzidine (TMB); however, it is to be understood that substrates other than TMB may also be used. Horseradish peroxidase catalyzes the oxidation of TMB when in the presence of hydrogen peroxide. The oxidized TMB may then be converted to the diimine form when subjected to acidic conditions. The diimine form of TMB may then be detected as discussed further below.

The addition of the substrate to one or more of the wells may be effected, for example, by dispensing a quantity of a solution comprising TMB and hydrogen peroxide to the one or more wells, allowing, through an incubation period, the enzymatic oxidation of TMB to occur, and then adding a suitable quantity of an acid, such as a sulfuric acid solution, to the one or more wells to convert the oxidized TMB to the diimine form and to terminate the oxidation reaction.

Method 11 may further comprise a step 27 of detecting the presence of the reacted substrate in one or more of the wells. In one embodiment, the detection of the reacted substrate may be determined through optical analysis, for example, by noting a colorimetric change in the contents of a well or by measuring the absorption of light in the contents of a well at one or more wavelengths indicative of the reaction of the substrate. Alternatively, in another embodiment, the detection of the reacted substrate may be determined through electrochemical analysis of the contents of the well, for example, using the electrochemical sensing element and chronoamperometry or voltammetry.

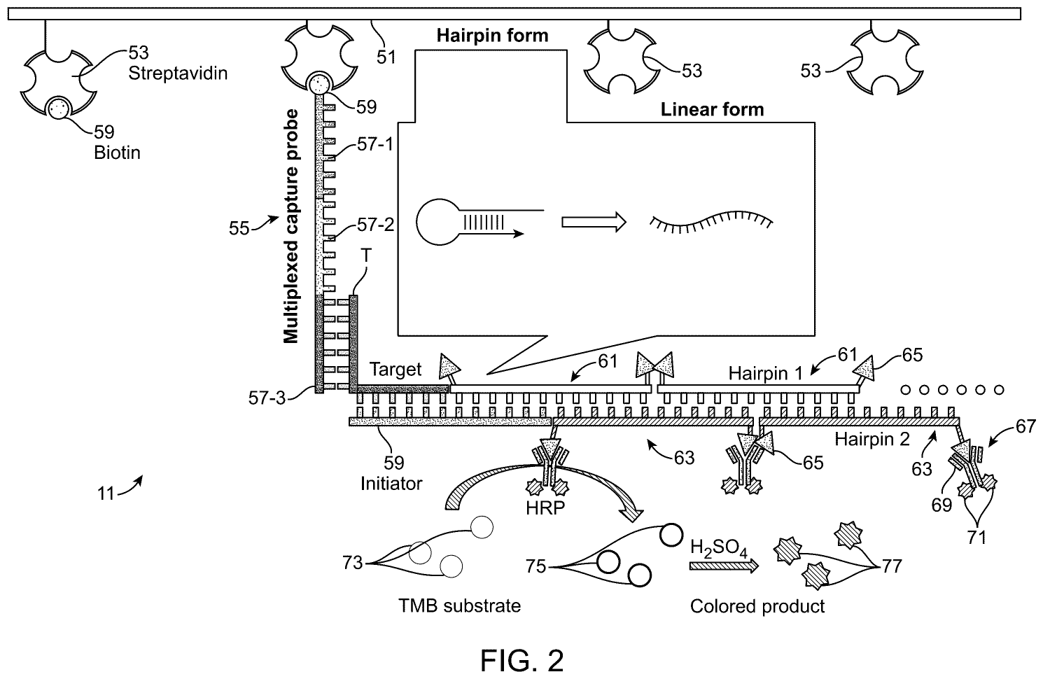

Referring now to FIG. 2, there is shown a schematic diagram of method 11. For simplicity and/or clarity, certain aspects of method 11 that are not critical to an understanding of the present invention are either not shown or described herein or are shown and/or described herein in a simplified manner.

As can be seen, FIG. 2 depicts the detection of a target allele of interest T. To this end, method 11 may involve providing a support 51, to which one or more streptavidin molecules 53 have been applied. Although support 51 is shown in FIG. 2 as a planar structure, support 51 may comprise a multi-well plate or may comprise an electrochemical sensing element comprising one or more electrodes associated with a structure (not shown) forming a well or may comprise some other type of structure having one or more analysis sites.

Method 11 may further involve immobilizing a multiplexed capture probe 55 to support 51 in one or more of the wells or analysis sites thereof. Multiplexed capture probe 55 may comprise a single-stranded nucleotide sequence having a plurality of capture sequences 57-1 through 57-3, each of which is specific for hybridization to a different target allele of interest. Multiplexed capture probe 55 may additionally comprise a biotin molecule 59 at its 5′-end to permit the remainder of multiplexed capture probe 55 to be securely coupled to a streptavidin molecule 53. Once multiplexed capture probe 55 has been immobilized on support 51 via streptavidin-biotin binding, multiplexed capture probe 55 may be exposed to a sample so that any target alleles of interest in the sample may be captured by one or more of the corresponding capture sequences 57-1 through 57-3 of multiplexed capture probe 55.

As shown in FIG. 2, a target allele of interest T has been captured by capture sequence 57-3 of multiplexed capture probe 55, with a first portion of target allele of interest T hybridizing to capture sequence 57-3 and with a second portion of target allele of interest T not hybridizing to capture sequence 57-3 or to any other portion of multiplexed capture probe 55.

Method 11 may further involve adding an initiator 59 to the well or analysis site containing multiplexed capture probe 55. In the embodiment shown, initiator 59 is designed to have a first portion that hybridizes to the second portion of target allele of interest T and a second portion that hybridizes to a first type of hairpin molecule 61. As can be appreciated, in those instances in which a target allele of interest T is not present in the sample, the addition of initiator 59 to the well or analysis site will be of no consequence as there will be nothing to which initiator 59 may hybridize.

Method 11 may further involve adding to the well or analysis site one or more copies of a first type of hairpin molecule 61 and one or more copies of a second type of hairpin molecule 63. Exposure of initiator 59 to a first copy of hairpin molecule 61 causes the first copy of hairpin molecule 61 to open and allows a first portion of the first copy of hairpin molecule 61 to hybridize to the second portion of initiator 59. The opening of the first copy of hairpin molecule 61, in turn, causes a first copy of hairpin molecule 63 to open and allows a first portion of the first copy of hairpin molecule 63 to hybridize to a second portion of the first copy of hairpin molecule 61. The opening of the first copy of hairpin molecule 63, in turn, causes a second copy of hairpin molecule 61 to open and allows a first portion of the second copy of hairpin molecule 61 to hybridize to a second portion of the first copy of hairpin molecule 63. This process, or hybridization chain reaction, may repeat one or more times. One or both of hairpin molecules 61 and 63 may further comprise one or more labels. In the present embodiment, a digoxigenin (DIG) label 65 may be coupled to each of 5′-end and 3′-end of the nucleotide sequence of each of hairpin molecules 61 and 63.

As can be appreciated, even though multiplexed capture probe 55 is capable of binding three different targets, one for each of capture sequences 57-1 through 57-3, the selective use of an initiator 59 that binds to only one of the three captured targets enables one to control if and when a hybridization chain reaction occurs.

Method 11 may further comprise adding an enzyme-linked antibody 67 to the well or analysis site. Enzyme-linked antibody 67 may comprise an anti-DIG antibody 69 that is designed to hybridize to the digoxigenin (DIG) label 65 on hairpin molecules 61 and 63. In addition, enzyme-linked antibody 67 may comprise an enzyme 71 conjugated to anti-DIG antibody 69. In the present embodiment, enzyme 71 may comprise a horseradish peroxidase (HRP) molecule or complex. As noted further below, horseradish peroxidase may be used to catalyze the oxidation of a substrate, and the occurrence of such oxidation may then be detected. Notwithstanding the above, it is to be understood that enzymes other than horseradish peroxidase may be used.

Method 11 may further comprise adding a substrate for enzyme 71. In the present embodiment, the substrate may comprise 3,3′,5,5′-tetramethylbenzidine (TMB) 73. Horseradish peroxidase catalyzes the oxidation of TMB when in the presence of hydrogen peroxide to form an oxidized TMB 75. The oxidized TMB 75 may then be converted to the diimine form 77 when subjected to acidic conditions (e.g., addition of sulfuric acid). The diimine form 77 of TMB may then be detected by optical analysis, for example, by noting a colorimetric change in the well or at the analysis site or by measuring the absorption of light in the well or at the analysis site. Alternatively, in another embodiment, the detection of the reacted substrate may be determined through electrochemical analysis at the well or analysis site, for example, using an electrochemical sensing element and chronoamperometry or voltammetry.

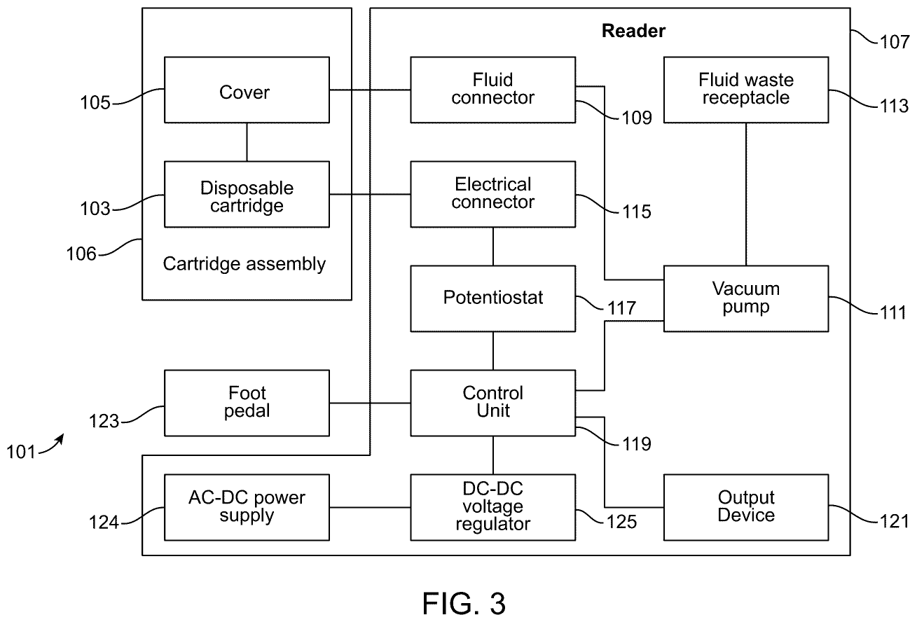

Referring now to FIG. 3, there is schematically shown one embodiment of a system for use in detecting one or more alleles of interest in a sample, the system being constructed according to the present invention and being represented generally by reference numeral 101. For simplicity and/or clarity, certain aspects of system 101 that are not critical to an understanding of the present invention are either not shown or described herein or are shown and/or described herein in a simplified manner.

System 101 may comprise a disposable cartridge 103. In at least one embodiment, disposable cartridge 103 may comprise a support that is shaped to define a plurality of discrete wells. For example, disposable cartridge 103 may comprise four separate wells. In addition, disposable cartridge 103 may further comprise a corresponding plurality of electrochemical sensing elements, with one electrochemical sensing element being associated with each well. In other words, disposable cartridge 103 may comprise four electrochemical sensing elements, with a different electrochemical sensing element being associated with each of the four wells. In at least one embodiment, the electrochemical sensing element may be at least partially coated with streptavidin and may comprise a working electrode, a counter electrode, and a reference electrode. The one or more electrodes of the electrochemical sensing element may be screen-printed electrodes, and each of the screen-printed electrodes may be formed using a carbon ink. Preferably, disposable cartridge 103 is suitably constructed so that steps 15, 17, 19, 21, 23 and 25 of method 11 may be performed in the wells of disposable cartridge 103. Disposable cartridge 103 is intended to be a single-use device. In at least one embodiment, one or more portions of disposable cartridge 103 may be partially or completely transparent.

System 101 may further comprise a cover 105. Cover 105, which may be disposable, may be removably mounted on top of disposable cartridge 103 so that disposable cartridge 103 and cover may jointly form a cartridge assembly 106. Cover 105 may serve to close the wells of disposable cartridge 103 and may be mounted on disposable cartridge 103 after performing step 25 of method 11 but before performing step 27 of method 11. Cover 105 may be constructed to include one or more fluid outlets, which may be used to be used to drain the fluid from cartridge assembly 106 after step 27 of method 11 has been performed. In at least one embodiment, cover 105 may be partially or completely transparent.

System 101 may further comprise a reader 107. Reader 107 may be used to perform the electrochemical analysis of the contents of the wells of disposable cartridge 103 in conjunction with the electrochemical sensing elements of disposable cartridge 103. Reader 107 may be designed to be a portable, multi-use item and may be cleaned, reconditioned and/or reset between uses.

Reader 107 may comprise a fluid connector 109. Fluid connector 109, which may include one or more fluid inlets adapted to mate with the fluid outlets of cover 105, may be coupled to a vacuum pump 111. In this manner, operation of vacuum pump 111 may cause the fluid in cartridge assembly 106 to be drained therefrom. A waste receptacle 113, which may be disposable, may be removably coupled to vacuum pump 111 and may be used to collect the fluid that is drained from cartridge assembly 106.

Reader 107 may further comprise one or more electrical connectors 115. Electrical connectors 115 may be constructed to couple the output ends of the electrochemical sensing elements of disposable cartridge 103 to a potentiostat 117. In at least one embodiment, potentiostat 117 may include four separate modules, and each of the four separate modules of potentiostat 117 may be electrically coupled via an electrical connector 115 to a different electrochemical sensing element of disposable cartridge 103. Potentiostat 117 may be used to perform an electrochemical analysis of a sample using, for example, chronoamperometry or voltammetry.

Reader 107 may further comprise a control unit 119. Control unit 119, which may comprise a central processing unit of a computer or the like and which may be equipped with suitable software, such as PSTrace software (PalmSens BV, Houten, The Netherlands), may be electrically coupled to potentiostat 117 in such a way as to control the operation of potentiostat 117 and to process and to evaluate the signals obtained from potentiostat 117. Control unit 119 may also be electrically coupled to vacuum pump 111 to control its operation.

Reader 107 may further comprise an output device 121. Output device 121, which may be, for example, a monitor or a printer, may be coupled to control unit 119 and may be used to display operating instructions for reader 107 and/or the results of any electrochemical analysis performed using potentiostat 117 and control unit 119.

Reader 107 may further comprise a foot pedal 123. Foot pedal 123 may be coupled to control unit 119 in such a manner that operation of foot pedal 123 may be used to cause the actuation of vacuum pump 111.

Reader 107 may further comprise an AC-DC power supply 124 and a DC-DC voltage regulator 125. AC-DC power supply 124 may be connectable to an AC power source, such as a wall outlet, and may be used to convert alternating current into direct current. DC-DC voltage regulator 125, which may be coupled to AC-DC power supply 124 and to control unit 119, may be used to convert the direct current voltage from AC-DC power supply 124 into a voltage more suitable for control unit 119.

Some or all of the components of reader 107, except for foot pedal 123, may be contained within a housing.

As can be appreciated, reader 107 may be configured so that its operation is largely automated. In other words, reader 107 may be configured so that, after coupling cartridge assembly 106 to reader 107, reader 107 may perform an electrochemical analysis of the contents of cartridge assembly 106. After such analysis has been conducted, actuation of foot pedal 123 may cause the fluid contents of cartridge assembly 106 to be disposed of in fluid waste receptacle 113, which, itself, may be disposed of or drained between one or more uses.

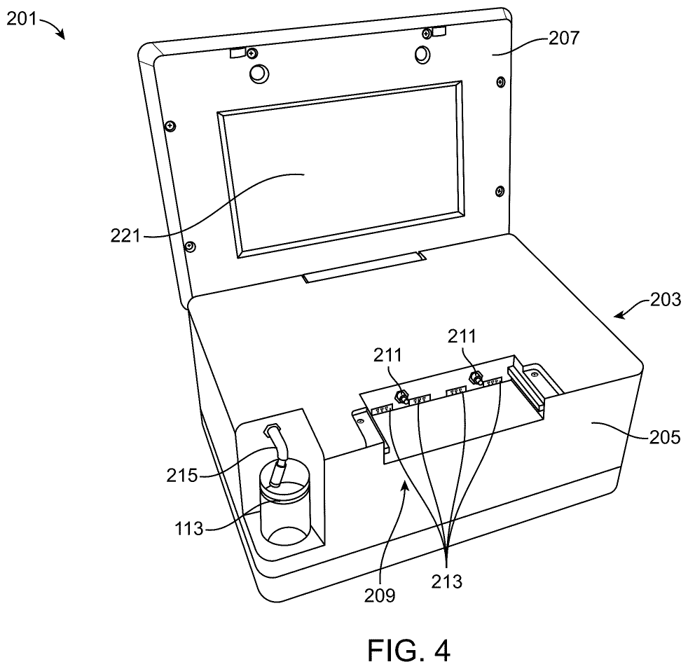

Referring now to FIG. 4, there is shown a perspective view of a physical manifestation of the reader shown in FIG. 3, the reader being constructed according to the present invention and being represented generally by reference numeral 201. For simplicity and/or clarity, certain aspects of reader 201 that are not critical to an understanding of the present invention are either not shown or described herein or are shown and/or described herein in a simplified manner.

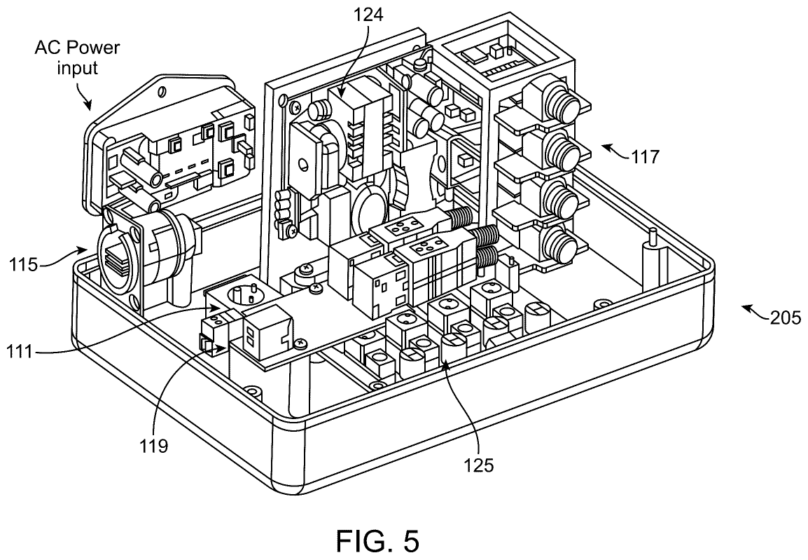

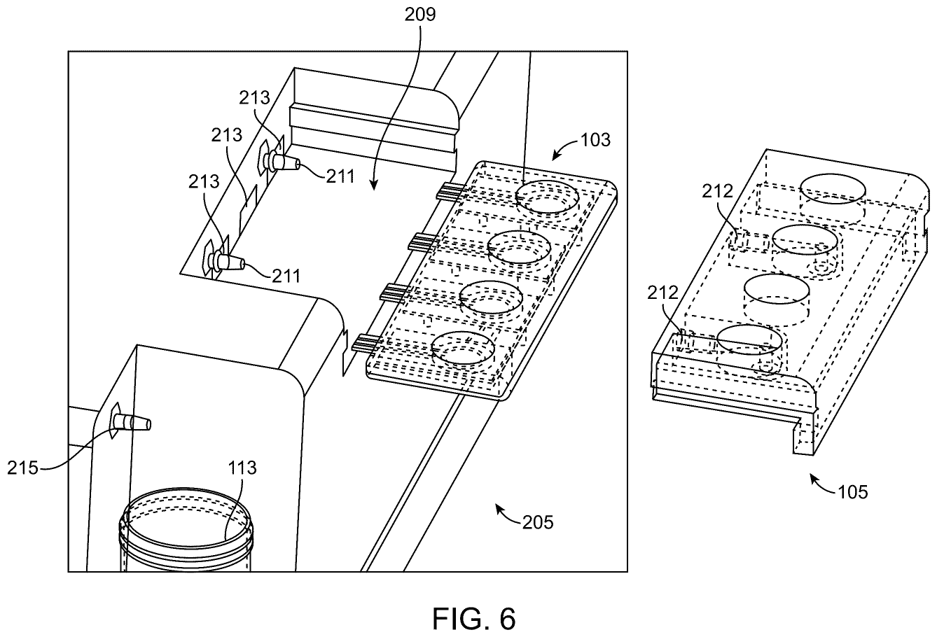

Reader 201 may comprise a housing 203. Housing 203, in turn, may comprise a base portion 205, and a cover portion 207. Base portion 205 may be configured to contain various components, such as, for example, vacuum pump 111, potentiostat 117, control unit 119, AC-DC power supply 124, and DC-DC voltage regulator 125 (see FIG. 5). Base portion 205 may also be shaped to include a shelf 209, which may be configured to matingly receive cartridge assembly 106. In addition, base portion 205 may also house fluid connector 109, whose inlets 211 may protrude from base portion 205 to mate with conduits 212 formed in cover 105 of cartridge assembly 106 (see FIGS. 4 and 6). Electrical connector 115, which is housed within base portion 205, may have inlets 213 that are accessible through openings in base portion 205 for mating with the electrodes of cartridge assembly 106 (see FIGS. 4 and 6). Further, base portion 205 may be configured to receive fluid waste receptacle 113, which may receive fluid waste pumped by vacuum 111 through an outlet 215 protruding from an opening in base portion 205 (sees FIGS. 4 and 6).

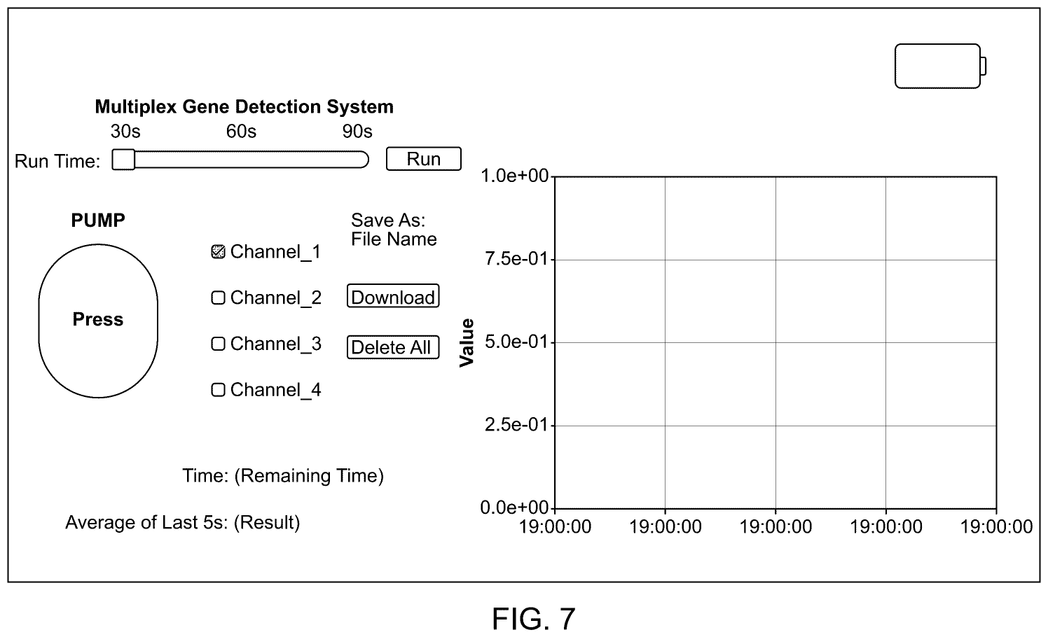

Cover portion 207 may be hingedly connected to base portion 205 and may contain a display 221 or other output device. FIG. 7 is an illustration of one embodiment of a graphical user interface suitable for use with display 221 of reader 201.

The following examples are given for illustrative purposes only and are not meant to be a limitation on the invention described herein or on the claims appended hereto.

### EXAMPLES

### Example 1: Materials and Equipment

All oligonucleotide sequences (i.e., the capture probe, the initiators, the first and second hairpin molecules (H1 and H2), and the targets for the CYP2D6 and CYP2C19 genes, namely, the allele variants CYP2D6*1, CYP2D6*10 and CYP2C19*17) were purchased from Integrated DNA Technologies, Inc. (Coralville, IA). The sequence information for the aforementioned oligonucleotides is presented below in TABLE I.

TABLE I

| Name | Sequence (5′→3′) |
|---|---|
| DNA | /5Biosg/CT GGT GAG TAG C |
| Capture | (SEQ ID NO: 10)/iSpC12/C |
| Probe | GGT GAG GCA GT (SEQ ID NO: 11)/ |
| | iSpC12/TAC ATC AGA GAT |
| | (SEQ ID NO: 1) |

CYP2D6 Sequences

| | |
|---|---|
| CYP2D6*1 | TCC TCG GGC CTA CTG CCT |
| target | CAC CG (SEQ ID NO: 2) |
| CYP2D6*1 | AGG CCC GAG GA TTA ACC |
| initiator | CAC GCC GAA TCC TAG ACT |
| | (SEQ ID NO: 3) |
| CYP2D6*10 | CTG GGC TGC ACG CTA CTC |
| target | ACC AG (SEQ ID NO: 4) |
| CYP2D6*10 | GTG CAG CCC AG TTA ACC |
| initiator | CAC GCC GAA TCC TAG ACT |
| | (SEQ ID NO: 5) |

CYP2C19 Sequences

| | |
|---|---|
| CYP2C19*17 | GTT CTC AAA GTA TCT CTG |
| target | ATG TA (SEQ ID NO: 6) |
| CYP2C19*17 | ACT TTG AGA ACT TAA CCC |
| initiator | ACG CCG AAT CCT AGA CT |
| | (SEQ ID NO: 7) |

Hairpin Molecule Sequences

| | |
|---|---|
| First | /5DigN/CAA AGT AGT CTA |
| Hairpin | GGA TTC GGC GTG (SEQ ID NO: 12)/ |
| Molecule | iC9Sp/TTA ACC CAC GCC GAA |
| (H1) | TCC TAG ACT/3Dig_N/ |
| | (SEQ ID NO: 8) |
| Second | /5DigN/CAC GCC GAA TCC |
| Hairpin | TAG ACT ACT TTG (SEQ ID NO: 13)/ |
| Molecule | iSpC12/AGT CTA GGA TTC GGC |
| (H2) | GTG GGT TAA/3Dig_N/ |
| | (SEQ ID NO: 9) |

Unconjugated anti-digoxigenin (i.e., anti-DIG) antibody (Catalog #200-002-156) and anti-DIG-mono horseradish peroxidase (HRP) conjugate (Catalog #200-032-156) were purchased from Jackson ImmunoResearch Labs (West Grove, PA). Goat anti-mouse polyHRP80 (Catalog #43-1005) was purchased from Biosynth International, Inc. (Gardner, MA). Streptavidin-coated 96-well plates and BLOCKER™ bovine serum albumin (BSA) 10% in phosphate buffered saline (PBS) (Catalog #37525) were purchased from Thermo Fisher Scientific, Inc. (Waltham, MA). 3,3′,5,5′-tetramethylbenzidine (TMB) substrate solution (Catalog #TMBS-1000-01) was purchased from BioFX Laboratories (Owing Mills, MD). 20λ PBS-T (PBS-Tween) buffer was purchased from Thermo Fisher Scientific, Inc. (Waltham, MA). 10×PBS buffer (pH 7.4), TE (Tris-EDTA) buffer (pH 8.0), and diethyl pyrocarbonate-treated water (DEPC-treated water) were purchased from Invitrogen Corporation (Carlsbad, CA). H₂SO₄ and all other reagents were purchased from Sigma-Aldrich (St. Louis, MO). Invitrogen's Ultrapure DNase-free, RNase-free DEPC-treated water (Catalog #4387937) was used in all studies. All commercial blood and bronchoalveolar (BAL) de-identified pooled fluids were purchased from BioIVT (Westbury, NY) and tested at Giner's Biosafety Level 2 lab facility (Newton, MA). A SpectraMax® iD3 plate reader from Molecular Devices (San Jose, CA) was used for colored TMB measurement. Metrohm Dropsens DRP-110 screen-printed carbon electrodes were purchased from Metrohm USA Inc. (Riverview, FL). An EMStat3+ USB-powered potentiostat was obtained from PalmSens BV (Houten, Netherlands).

Purchased DNA oligonucleotides were re-suspended in DEPC-treated H₂O and stored at −20° C. as DNA stocks. DNA probe immobilization and target hybridization buffers were prepared with 1×PBS buffer. In addition, 1×PBS-T buffer, 1×PBS buffer, and 1% BSA in 1×PBS buffer were obtained from buffer stock solutions. The unconjugated anti-DIG antibody and the goat anti-mouse polyHRP80 were diluted in 1% BSA in 1×PBS buffer to the desired concentration. H₂SO₄ stock solution was diluted with deionized water to obtain a 0.5 M H₂SO₄ solution. DEPC-treated water was used for all other solution preparations in the experiments.

### Example 2: Hybridization Chain Reaction (HCR) Assay Development

An enzymatic-reaction-based assay was developed to detect the presence of various human cytochrome P450 enzyme alleles in a sample. As shown schematically in FIG. 2, the assay begins by immobilizing one or more copies of a biotinylated, allele-recognizing capture probe on a streptavidin-coated plate, such immobilization of the capture probe on the plate being effected via streptavidin-biotin binding. In the present embodiment, each capture probe contains three allele-specific complementary nucleotide sequences, one directed at a CYP2D6*1 target allele, one directed at a CYP2D6*10 target allele, and one directed at a CYP2C19*17 target allele. In the event that a target allele is present in a sample that is contacted with the capture probe, a portion of the target allele hybridizes to the corresponding allele-specific complementary sequence of the capture probe. An allele-specific initiator that has a sequence that is complementary to another portion of the captured target allele is then contacted with and hybridizes to the captured target allele. The initiator also has a sequence that is complementary to a portion of the first hairpin molecule (i.e., H1); consequently, the initiator can bind both to the captured target allele and to the first hairpin molecule. Binding of the initiator to the first hairpin molecule causes the first hairpin molecule to open and permits a hybridization chain reaction to occur between the first hairpin molecule and the second hairpin molecule (i.e., H2). A digoxigenin tag is present on both 3′ end and 5′ end of each of the first and second hairpin molecules. The digoxigenin tag can then be detected by contacting the digoxigenin tag with anti-digoxigenin antibodies coupled to an enzyme, such as horseradish peroxidase, that causes a detectable change (e.g., colorimetric, electrochemical) in a substrate like 3,3′,5,5′-tetramethylbenzidine (TMB). Thus, the presence of a detectable change in the substrate can be used to indicate the presence of a particular allele variation in the sample being tested.

In a particular embodiment, the above-described assay was conducted using a streptavidin-coated 96-well plate (Thermo Fisher Scientific, Waltham, MA) in a volume of 100 μL. All DNA probes and targets were diluted to corresponding concentrations with 1×PBS buffer (diluted from 10×PBS buffer, Thermo Fisher Scientific, Waltham, MA). All incubation steps were conducted under ambient temperature at 500 RPM, followed by three rounds of 300 μL 1×PBS-T wash (diluted from 20×PBS-T buffer, Thermo Fisher Scientific, Waltham, MA), unless otherwise noted. Streptavidin-coated wells were first incubated with a 100 nM biotin-tagged capture probe for 20 minutes. After washing, a DNA target in corresponding concentrations was applied for a 30-minute incubation, followed by a 20-minute incubation with 200 nM initiator. Dual digoxin-tagged hairpin molecules were separately diluted to 500 nM in 2 mL tubes and incubated in a 95° C. metal bath for 5 minutes. The tubes were then placed on ice for proper folding for 5 minutes before they were removed to adjust to ambient temperature for at least 15 minutes. Ambient temperature hairpin molecules H1 and H2 were then combined and mixed well before being applied to wells for a 2-hour hybridization chain reaction incubation. For detection, Peroxidase IgG Fraction Monoclonal Mouse Anti-Digoxin (Jackson ImmunoResearch Labs, West Grove, PA) was diluted in a 1:12500 ratio with 1% bovine serum PBS buffer (diluted from 10% BSA in PBS, Thermo Fisher Scientific, Waltham, MA) before the antibody was applied to the wells and incubated for 30 minutes. TMB (BioFX Laboratories, Owing Mills, MD) at ambient temperature was then added to the wells and allowed to incubate for 10 minutes away from light. Without washing, 100 μL of 1M H₂SO₄ was applied to terminate the reaction, and the plate was imaged with a microplate reader to record absorbance at 450 nm.

### Example 3: Evaluation of Hybridization Chain Reaction with Initiator and Different Hairpins

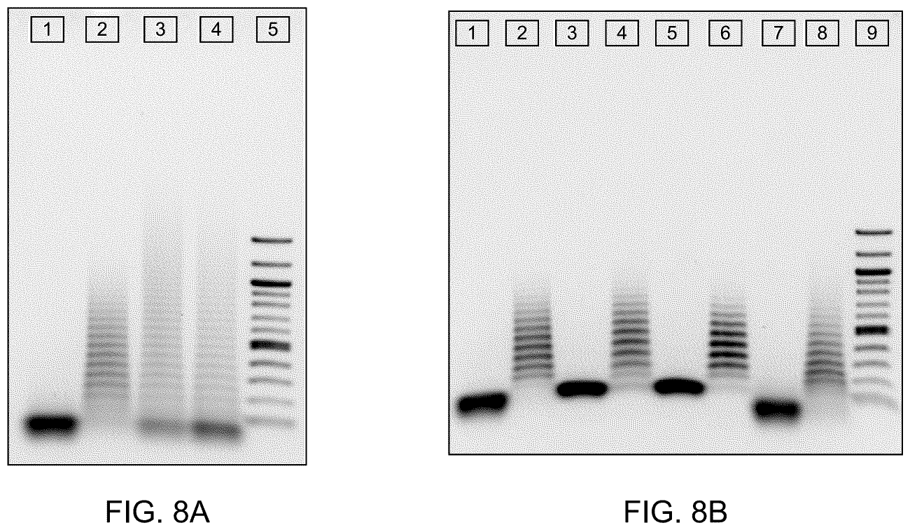

To evaluate the performance of the hybridization chain reaction (HCR) utilized in the present invention, an electrophoresis gel experiment was conducted using unmodified versions of hairpin molecules H1 and H2. More specifically, referring now to FIG. 8A, each of Lanes 1 to 4 of a 1% agarose gel were loaded with 250 nM of an unmodified version of H1 and 250 nM of an unmodified version of H2. Lane 2 additionally had 200 nM of the initiator for CYP2D6*10, Lane 3 additionally had 125 nM of the initiator for CYP2D6*10, and Lane 4 additionally had 85 nM of the initiator for CYP2D6*10. As hypothesized, Lane 2, which had the highest amount of initiator, had the highest rate of HCR, which was evidenced by the presence of a long band corresponding to HCR product and by the disappearance of the band corresponding to the unopened hairpins. Lanes 3 and 4 had lesser amounts of initiator than did Lane 2; hence, there were more unopened hairpins left over in Lanes 3 and 4 than in Lane 2, as evidenced by the darker hairpin band at the bottom of each of Lanes 3 and 4.

The gel of FIG. 8A confirms the operability of the CYP2D6*10 initiator with unmodified versions of H1 and H2. The operability of the CYP2D6*10 initiator was then tested with hairpins having different types of DIG-modifications. More specifically, the operability of the CYP2D6*10 initiator was tested with versions of H1 and H2 having a single DIG modification, with versions of H1 and H2 having dual DIG modifications but no spacer, with versions of H1 and H2 having dual DIG modifications and a loop spacer modification, and with unmodified versions of H1 and H2. Referring now to FIG. 8B, Lane 1 of a 1% agarose gel had a single DIG modification in each of H1 and H2 but no initiator whereas Lane 2 of the gel had a single DIG modification in each of H1 and H2 and also included 200 nM of CYP2D6*10 initiator. Lane 3 of the gel had dual DIG modifications in each of H1 and H2 but no loop spacer and no initiator whereas Lane 4 of the gel had dual DIG modifications in each of H1 and H2 and no loop spacer but also included 200 nM of CYP2D6*10 initiator. Lane 5 of the gel had dual DIG modifications in each of H1 and H2 and a loop spacer but no initiator whereas Lane 6 of the gel had dual DIG modifications in each of H1 and H2 and a loop spacer and also included 200 nM of CYP2D6*10 initiator. Lane 7 of the gel had unmodified H1 and unmodified H2 but no initiator whereas Lane 8 of the gel had unmodified H1 and unmodified H2 and also included 200 nM of CYP2D6*10 initiator. In other words, Lanes 1, 3, 5 and 7 had various types of hairpin molecules but without initiator whereas Lanes 2, 4, 6 and 8 not only had the hairpin molecules but also had initiator. As can be seen, the initiator was able to successfully initiate hybridization chain reaction for all of the different hairpin types.

### Example 4: Presence Test

After obtaining gel electrophoresis results that demonstrated the successful initiation of the hybridization extension of all hairpin variants tested, the capacity of the initiator to successfully initiate such hybridization extension in plate-based reactions was then investigated. To this end, a comparison was undertaken of the results obtained using 5 pM of CYP2D6*10 initiator with the full set of components, namely, capture probe, CYP2D6*10 target, and loop spacer hairpins, with the corresponding results obtained under conditions where one or more of the foregoing components were absent. TABLE II below details the components present in each sample tested, with “+” denoting the presence of the component in the sample and “−” denoting the absence of the component in the sample.

TABLE II

| | Sam- | Sam- | Sam- | Sam- | Sam- | Sam- |
|---|---|---|---|---|---|---|
| Component | ple 1 | ple 2 | ple 3 | ple 4 | ple 5 | ple 6 |
| Capture | + | + | + | + | + | − |
| probe | | | | | | |
| Target | − | + | + | + | + | + |
| Initiator | − | − | + | + | − | + |
| Hairpins | − | − | − | + | + | + |

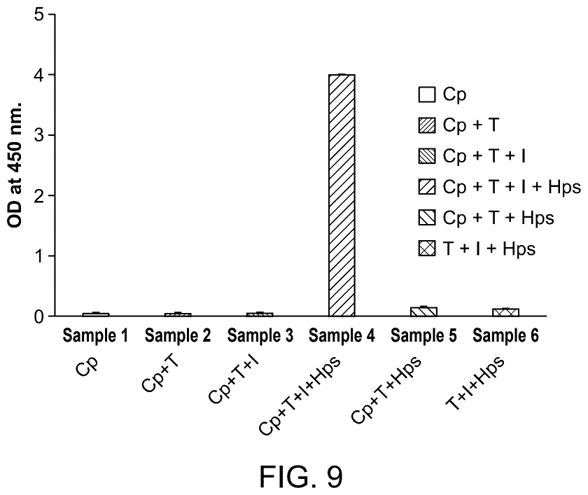

As can be seen in FIG. 9, hybridization chain reaction occurred when all essential reaction components were present (i.e., Sample 4) but did not occur when any components were missing (i.e., Samples 1-3 and Samples 5-6). The low signal intensity in setups lacking one or more of the assay components suggested minimal nonspecific binding and extension in the assay background.

### Example 5: Optimization of Buffer Combinations

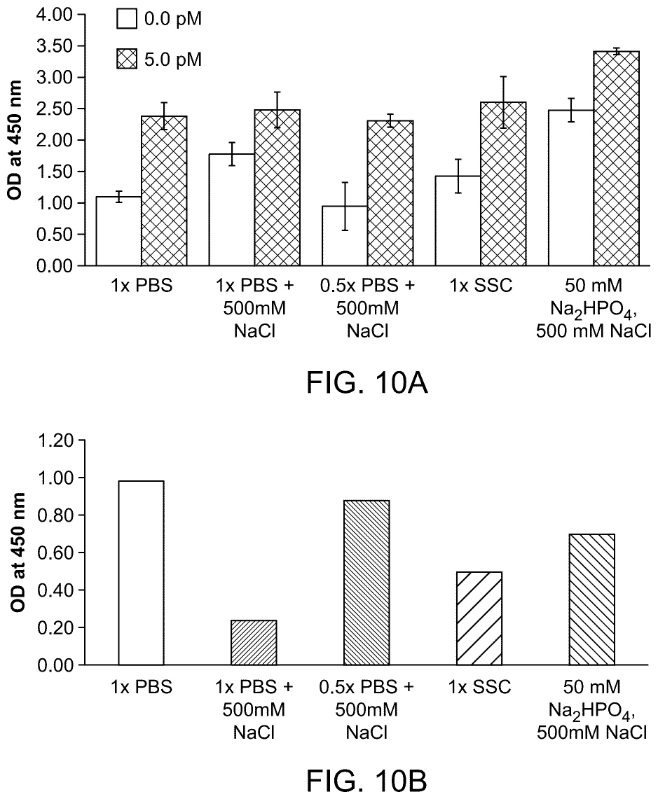

To determine which buffer condition is best suited for the hybridization chain reaction employed in the present invention, five different buffers were prepared and tested: (A) 1×PBS; (B) 1×PBS with 500 mM NaCl; (C) 0.5×PBS with 500 mM NaCl; (D) 1×SSC buffer; and (E) 50 mM Na₂HPO₄ with 500 mM NaCl. (See FIG. 10A, labeled A-E). The results showed that 5 pM CYP2D6*10 targets could be detected using all the buffers; however, each buffer produced varying signal intensity and background noise. The standard deviation for the 5 pM targets, the average signal for a 0 pM target, and the standard deviation for the 0 pM target were subtracted from the average signal for the 5 pM targets in all five buffers. The resulting values are shown in FIG. 10B, from which it can be seen that 1×PBS is the optimal buffer within the group tested.

### Example 6: Sensitivity Test

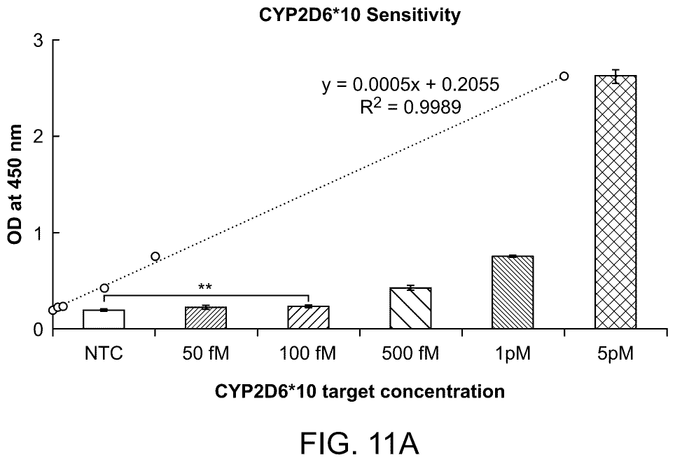

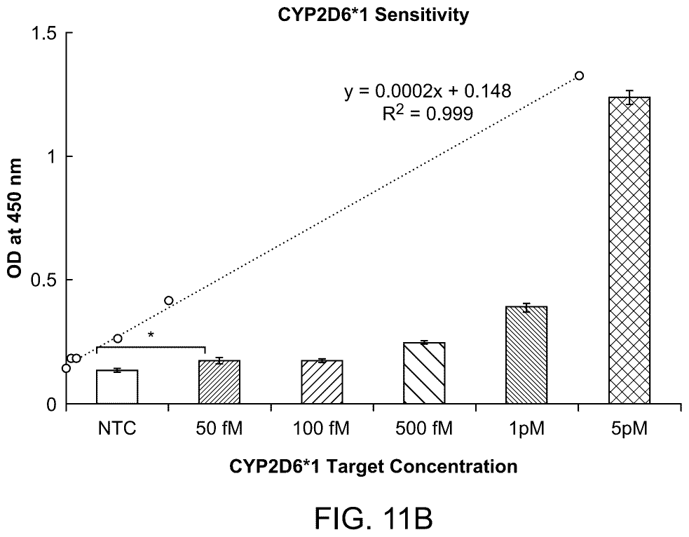

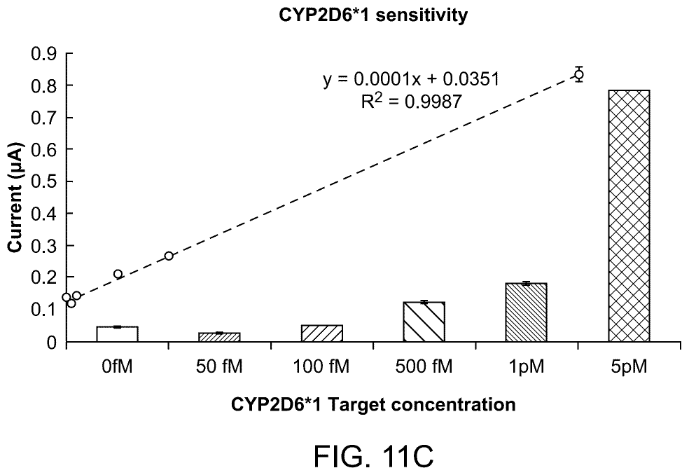

Following the determination of the optimal buffer condition (1×PBS) for the hybridization chain reaction of the present invention, the sensitivity of said hybridization chain reaction was assessed by performing an experiment with a range of target concentrations (50 fM to 5 pM) of allele CYP2D6*10. 5 pM was selected as the maximum concentration because optimization tests with the initiator for this target demonstrated that, at 5 pM, the optical density reaches 4 and becomes saturated (based on the limit of the instrument used). The results of the aforementioned testing are presented in FIG. 11A. The sensitivity of the assay of the present invention was significantly improved as compared to existing assays, with a limit of detection (LOD) for the target using the present assay being 100 fM, as compared to 10 pM for existing assays. A similar sensitivity test was performed for the CYP2D6*1 allele, and the results showed that sensitivity to detect was as low as 50 fM of the target (see FIG. 11B). In addition, electrochemical readings for CYP2D6*1 allele detection were obtained and demonstrated a linear correlation between the average electrical current (A) measured over the last five seconds and the target oligonucleotide concentration (see FIG. 11C).

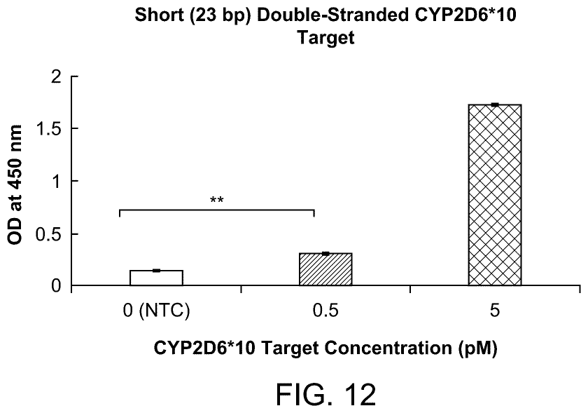

Additionally, to further validate the present assay, the detection of double-stranded DNA (dsDNA) targets was also tested. Due to the challenge of binding the capture probe to double-stranded targets, the capture probe and the target were mixed, with the mixture then being heated at 95° C. for 10 minutes, then being allowed to cool to room temperature for 10 minutes, and then being added to the reaction to ensure that the dsDNA was denatured before being exposed to the capture probe. The results showed that dsDNAs could be detected in 500 fM concentrations of allele CYP2D6*10 (see FIG. 12). This is as compared to an LOD of 60 pM (n=3) for dsDNA using existing techniques. The detection of dsDNA is an indicator of the operability of the present assay in the detection of clinical genomic DNA (gDNA) samples.

### Example 7: Specificity Test

To further validate the specificity of the present assay, specificity tests were conducted in both 1×PBS buffer and under preclinical 5% serum conditions. Four different target oligo combinations to be detected by a 12C spacer DNA capture probe were used, which capture probe could bind with all three targets, respectively. The CYP2D6*10 initiator was used, which specifically binds to the CYP2D6*10 target oligo to initiate the extension. The four target combinations were as follows: (i) pure 1×PBS buffer with no target; (ii) 5 pM of CYP2D6*10 target, (iii) 5 nM each of CYP2D6*1 and CYP2C19*3 targets, and (iv) a mixture of 5 pM CYP2D6*10 target, 5 nM CYP2D6*1 target, and 5 nM CYP2C19*3 target.

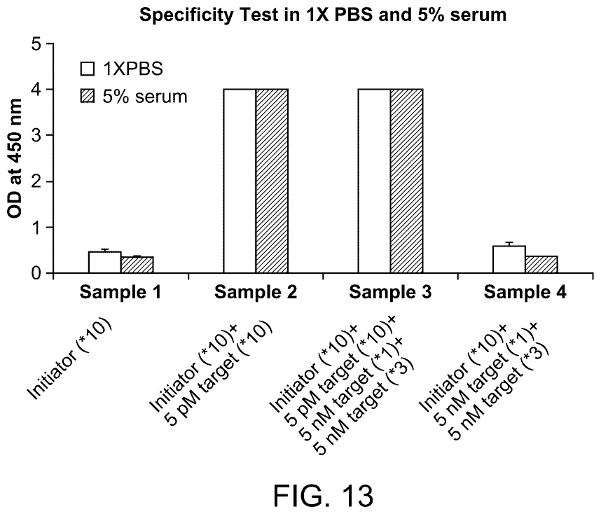

As shown in FIG. 13, the results demonstrated high assay specificity in both 1×PBS and 5% serum conditions, as a significant signal increase was observed only when the CYP2D6*10-specific initiator co-existed with the captured CYP2D6*10 target. The minimal difference between sets with or without a 1000-fold excess of CYP2D6*1 and CYP2C19*3 interference suggested that the capture capacity of the multiplexed capture probe remained unaffected when multiple targets were bound to the same probe. Additionally, the comparable signal intensity between the buffer and 5% serum conditions demonstrated that our assay has high applicability in preclinical settings.

TABLE III

| Component | Sample 1 | Sample 2 | Sample 3 | Sample 4 |
|---|---|---|---|---|
| *10 Initiator | + | + | + | + |
| 5 pM *10 target | − | + | + | − |
| 5 nM *1 target | − | − | + | + |
| 5 nM *3 target | − | − | + | + |

### Example 8: Additional Testing

A physical embodiment of a reader corresponding to reader 201 was assembled and included a RASPBERRY PI 5™ CPU (Raspberry Pi Holdings, Cambridge, UK) connected to an ARDUINO™ microcontroller board (BCMI, Monza, Italy), which microcontroller board reports battery voltage, pump status, and the active channel to the RASPBERRY PI 5™ CPU every 10 seconds. The RASPBERRY PI 5™ CPU, in turn, sends commands to the ARDUINO™ microcontroller board based on user input. A battery level indicator was integrated into a user interface and appears in its top-right corner (see FIG. 7). The 12V to 16.8V range of the battery is converted into a 0-100% scale, and when the charge drops below 30%, the battery icon turns red to alert the user.

Additionally, the RASPBERRY PI 5™ CPU executes commands through a pre-configured MethodSCRIPT™ scripting language generated by PSTrace software (PalmSens BV, Houten, The Netherlands), which is stored locally and allows users to modify runtime settings. The application displays real-time data on a custom chart, with interactive features that enable users to zoom, drag, and adjust the view using two-finger gestures. The main screen also provides key metrics, including the remaining runtime and the average value from the last five seconds. An enlarged image of the custom user interface is shown in FIG. 7.

Electrochemical testing using the prototype. For electrochemical detection, the above-described prototype integrates an EmStat 4-module USB-powered potentiostat (PalmSens BV, Houten, Netherlands), with readings collected and analyzed by a custom-developed program running on a RASPBERRY PI 5™ CPU with touchscreen capability. To assess the accuracy and comparability of the electrochemical readings of the prototype reader against those obtained using the official PalmSens software, PSTrace, on a Windows PC, we measured the same set of samples with varying anti-DIG-HRP concentrations using three setups: a benchtop standard plate reader, an EmStat 3-module potentiostat (PalmSens BV, Houten, Netherlands) connected to a Windows PC with the aforementioned PSTrace software, and our prototype equipped with an EmStat 4-module potentiostat.

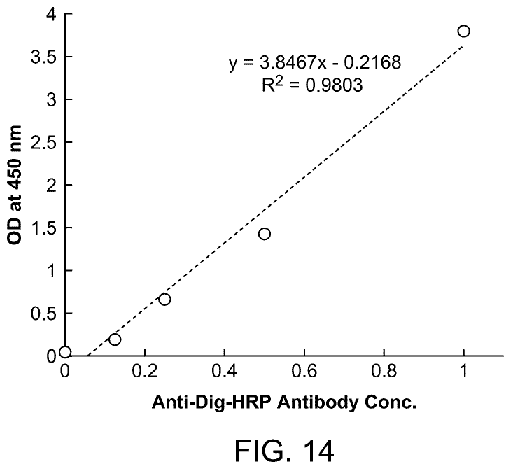

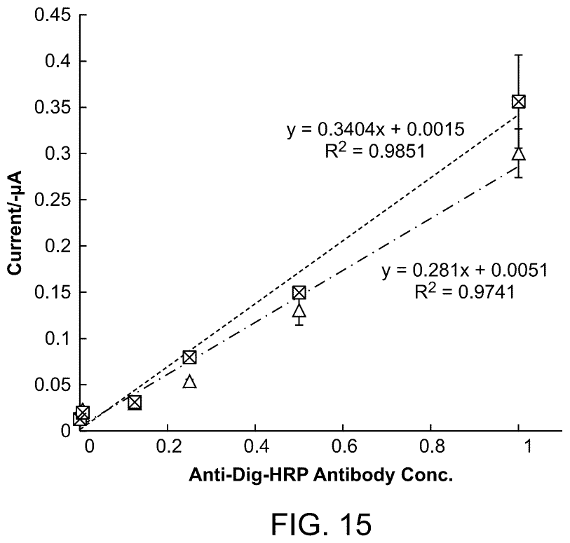

As shown in FIG. 14, the optical density (OD) at 450 nm increased along with rising anti-DIG-HRP antibody concentrations, as a higher antibody concentration resulted in a greater color change of the TMB substrate when incubated for the same duration. A strong correlation (R²>0.98) between antibody concentration and signal value were also observed when detected via both PSTrace on Windows PC and our own program on the prototype (see FIG. 15). The excellent linearity of the electrochemical reading obtained from the prototype and its corresponding HRP-tagged antibody concentrations confirmed that the circuit and the setup of the prototype did not compromise the performance of the EmStat 4-module. Moreover, the accuracy of our program in collecting and analyzing data from the module further validates its proper functionality.

Some desirable features, attributes, aspects, innovations and/or advantages of one or more embodiments of the present invention may include one or more of the following:
Hybridization chain reaction may be utilized in accordance with the present invention to develop a multiplexed, reprogrammable, and inexpensive technique for detecting common genetic variations in CYP2D6 and CYP2C19 cytochrome P450 enzymes under ambient temperature.
The present invention enables the development of a cytochrome P450 genetic variation detector that requires minimal specialized equipment and professional labor while still serving as a reliable indicator for personalizing treatment plans in clinical practice.
The present invention addresses the need for efficient, multiplexed SNP (single nucleotide polymorphism) detection with a PCR-free assay. This compact, cost-effective assay may operate at the point of care (POC) and may be used to detect multiple gene biomarkers in biofluids, such as blood. The present technique may utilize an enzymatic system with capture probes, initiator probes, and amplification hairpin probes to achieve high sensitivity and up to attomolar-level detection.
By leveraging, in one embodiment, a biotin-modified capture probe and dual-digoxigenin probes, the present invention may offer up to 100× signal amplification, up to 95% sensitivity, and up to attomolar-level detection (5-100 DNA copies/mL). The sample-to-result turnaround time of the present technique can be as short as only a few hours (e.g., ˜3.5 hours) or less. The present technique, optimized through comprehensive bioinformatic analysis and experimental adjustments, may be used to detect, for example, two target genes and their allele variations with sensitivity improved to femtomolar levels and potentially further enhanced by integrating a novel cluster of horseradish peroxidase molecules.
Validation of the accuracy of the present technique may be confirmed by the absence of false positives using all probes for the CYP2D6*10 allele.
The present technique achieves a substantial improvement in sensitivity, reducing the limit of detection (LOD) for CYP2D6*10 and CYP2D6*1 alleles to 50 fM.
The present technique may be used to detect double-stranded DNAs (dsDNAs) at concentrations as low as 500 fM using preclinical sample models.
The robustness of the present technique may be demonstrated by the successful detection of DNA targets spiked in biological fluids (5% serum) and 1×PBS, even in the presence of non-specific or scrambled targets.
The use of a spacer (such as iC9Sp, a non-nucleotide, aliphatic chain consisting of 9 carbon atoms, or ISpC12, a similar spacer consisting of 12 carbon atoms) inserted between two nucleotide residues, preferably at or near the middle of a hairpin nucleotide sequence, may provide flexibility and/or distance within the aforementioned sequence. Such a spacer has been found to improve the sensitivity and performance of the hairpin molecule in hybridization chain reaction. Notwithstanding the above, the hairpin molecule could be used without such a spacer.

The embodiments of the present invention described above are intended to be merely exemplary and those skilled in the art shall be able to make numerous variations and modifications to it without departing from the spirit of the present invention. All such variations and modifications are intended to be within the scope of the present invention.

What is claimed is:

1. A method for detecting the presence of one or more alleles of interest in a sample, the method comprising the steps of:
a) providing an immobilized capture probe on a support, the immobilized capture probe comprising a first nucleotide capture sequence and a second nucleotide capture sequence, the first nucleotide capture sequence designed to hybridize specifically with a first portion of a first allele of interest, the second nucleotide capture sequence designed to hybridize specifically with a first portion of a second allele of interest, the second allele of interest being different from the first allele of interest;
b) exposing the immobilized capture probe to a sample and removing any non-specific binding, whereby, if the sample contains the first allele of interest, the first portion of the first allele of interest specifically hybridizes to the first nucleotide sequence, and/or if the sample contains the second allele of interest, the first portion of the second allele of interest specifically hybridizes to the second nucleotide sequence, whereas, if the sample contains neither the first allele of interest nor the second allele of interest, nothing specifically hybridizes to the first nucleotide sequence or the second nucleotide sequence;
c) exposing the immobilized capture probe, and any first and second alleles of interest bound thereto, to a first initiator and removing any non-specific binding, wherein a first portion of the first initiator is designed to hybridize specifically to one of a second portion of the first allele of interest and a second portion of the second allele of interest;
d) exposing the immobilized capture probe, and any initiator bound thereto, to first and second hairpin molecules and removing any non-specific binding, wherein the first and second hairpin molecules are designed to undergo a hybridization chain reaction when actuated by the first initiator, and wherein at least one of the first and second hairpin molecules is coupled to a tag; and
e) determining the presence of the tag.

2. The method as claimed in claim 1 wherein the one or more alleles of interest include one or more alleles of cytochrome P450 genes.

3. The method as claimed in claim 2 wherein the cytochrome P450 genes include one or more of CYP2D6 and CYP2C9.

4. The method as claimed in claim 1 wherein the one or more alleles of interest are selected from the group consisting of an allele containing a CYP2D6*1 single nucleotide polymorphism, an allele containing a CYP2D6*10 single nucleotide polymorphism, and an allele containing a CYP2C19*17 single nucleotide polymorphism.

5. The method as claimed in claim 1 wherein the immobilized capture probe further comprises a third nucleotide capture sequence, the third nucleotide capture sequence designed to hybridize specifically with a first portion of a third allele of interest, the third allele of interest being different from the first and second alleles of interest, the initiator being designed to hybridize specifically to one of a second portion of the first allele of interest, a second portion of the second allele of interest, and a second portion of the third allele of interest.

6. The method as claimed in claim 1 wherein the support comprises a first well and wherein at least one copy of the immobilized capture probe is disposed in the first well.

7. The method as claimed in claim 6 wherein the support further comprises a second well, the method further comprising repeating steps (a) through (e) for the second well, except that a second initiator is used instead of the first initiator in the second well, the second initiator being designed to hybridize specifically to the other of the second portion of the first allele of interest and the second portion of the second allele of interest.

8. The method as claimed in claim 1 wherein the support comprises an electrochemical sensing element.

9. The method as claimed in claim 8 wherein the support further comprises a well, the electrochemical sensing element being associated with the well.

10. The method as claimed in claim 1 wherein the step of determining the presence of the tag comprises exposing the tag to an enzyme-linked antibody that is designed to specifically bind to the tag and removing any non-specific binding, exposing the enzyme-linked antibody to a substrate that undergoes a detectable change when exposed to the enzyme-linked antibody, and detecting the detectable change in the substrate.

11. The method as claimed in claim 10 wherein the detectable change in the substrate is determined by optical analysis.

12. The method as claimed in claim 10 wherein the detectable change in the substrate is determined by electrochemical analysis.

13. The method as claimed in claim 1 wherein the first hairpin molecule includes a first hairpin nucleotide sequence, wherein the second hairpin molecule includes a second hairpin nucleotide sequence and wherein the first hairpin molecule further includes a first spacer at or near a midpoint of the first hairpin nucleotide sequence and/or the second hairpin molecule further includes a second spacer at or near a midpoint of the second hairpin nucleotide sequence.

14. A method for detecting the presence of a cytochrome P450 allele of interest in a sample, the method comprising the steps of:
a) providing an immobilized capture probe on a support, the immobilized capture probe comprising a first nucleotide capture sequence, the first nucleotide capture sequence designed to hybridize specifically with a first portion of a cytochrome P450 allele of interest;
b) exposing the immobilized capture probe to a sample and removing any non-specific binding, whereby, if the sample contains the cytochrome P450 allele of interest, the first portion of the cytochrome P450 allele of interest specifically hybridizes to the first nucleotide sequence whereas, if the sample does not contain the cytochrome P450 allele of interest, nothing specifically hybridizes to the first nucleotide sequence;
c) exposing the immobilized capture probe, and any cytochrome P450 allele of interest bound thereto, to a first initiator and removing any non-specific binding, wherein a first portion of the first initiator is designed to hybridize specifically to the cytochrome P450 allele of interest;
d) exposing the immobilized capture probe, and any first initiator bound thereto, to first and second hairpin molecules and removing any non-specific binding, wherein the first and second hairpin molecules are designed to undergo a hybridization chain reaction when actuated by the first initiator, and wherein at least one of the first and second hairpin molecules is coupled to a tag; and
e) determining the presence of the tag.

15. The method as claimed in claim 14 wherein the cytochrome P450 allele of interest is an allele of one of CYP2D6 and CYP2C9.

16. The method as claimed in claim 15 wherein the cytochrome P450 allele of interest is selected from the group consisting of an allele containing a CYP2D6*1 single nucleotide polymorphism, an allele containing a CYP2D6*10 single nucleotide polymorphism, and an allele containing a CYP2C19*17 single nucleotide polymorphism.

17. The method as claimed in claim 14 wherein the support comprises a first well and an electrochemical sensing element associated with the well and wherein the immobilized capture probe is disposed in the first well.

18. The method as claimed in claim 14 wherein the step of determining the presence of the tag comprises exposing the tag to an enzyme-linked antibody that is designed to specifically bind to the tag and removing any non-specific binding, exposing the enzyme-linked antibody to a substrate that undergoes a detectable change when exposed to the enzyme-linked antibody, and detecting the detectable change in the substrate.

19. The method as claimed in claim 18 wherein the detectable change in the substrate is determined by optical analysis.

20. The method as claimed in claim 18 wherein the detectable change in the substrate is determined by electrochemical analysis.

21. A system for use in electrochemically detecting an allele of interest in a sample, the system comprising:
(a) a cartridge assembly, the cartridge assembly comprising
(i) a cartridge, the cartridge comprising one or more wells and further comprising a corresponding electrochemical sensing element associated with each of the one or more wells, and
(ii) a cover, the cover being removably mounted on the cartridge, the cover being shaped to include one or more fluid conduits in fluid communication with the one or more wells; and
(b) a reader for electrochemically detecting the presence of an analyte of interest in each of the one or more wells, the analyte of interest being indicative of the presence of the allele of interest, the reader comprising
(i) a potentiostat;
(ii) one or more electrical connectors for electrically connecting the one or more electrochemical sensing elements to the potentiostat;
(iii) a control unit operably coupled to the potentiostat for operating the potentiostat and for analyzing signals generated by the potentiostat;
(iv) an output device operably coupled to the control unit for outputting results of analysis performed by the control unit;
(v) a pump, the pump being operably coupled to the control unit;
(vi) one or more fluid connectors operably coupled to the one or more fluid conduits in the cover and to the pump;
(vii) a fluid waste receptacle operably coupled to the pump for receiving fluid removed from the one or more wells by the pump; and
(viii) a power supply.

22. The system as claimed in claim 21 wherein the potentiostat has one or more modules, each module corresponding to a different one of the one or more electrochemical sensing elements.

23. The system as claimed in claim 21 further comprising a foot pedal, the foot pedal being operably coupled to the control unit to actuate operation of the pump.

24. The system as claimed in claim 21 wherein each electrochemical sensing element comprises a working electrode, a counter electrode and a reference electrode.

25. The system as claimed in claim 24 wherein one or more of the working electrode, the counter electrode and the reference electrode is screen-printed using a carbon ink.

26. The system as claimed in claim 21 wherein the output device is a monitor.

27. The system as claimed in claim 21 further comprising a housing, wherein the housing is shaped to include a shelf, the shelf being configured to matingly receive the cartridge assembly in such a way as to connect the one or more fluid conduits of the cover to the one or more fluid connectors of the reader and to connect the one or more electrochemical sensing elements of the cartridge to the one or more electrical connectors of the reader.
